(19) United States
(12) Patent Application Publication
(10) Pub. No.: US 2003/0073607 A1
(43) Pub. Date: Apr. 17, 2003

(54) Pro-perfume compositions

(75) Inventors: Johan Smets, Lubbeek (BE); Rafael Trujillo, Mason, OH (US); Abdennaceur Fredj, Loveland, OH (US); Daniel Jerome White JR., West Chester, OH (US); Robert Walter Boswell JR., Hamilton, OH (US); Gordon Ridley, (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/141,730

(22) Filed: May 9, 2002

Related U.S. Application Data
(60) Provisional application No. 60/290,280, filed on May 11, 2001.

(51) Int. Cl. A61K 7/46

(52) U.S. Cl. 512/27; 510/108

(57) ABSTRACT

Disclosed herein are pro-perfume compositions comprising the reaction product of a primary and/or secondary amine compound with a combination of a perfume ketone component and a high molecular weight, high boiling point perfume aldehyde component. Pro-perfumes made from this selected type of reaction product provide a multi-odor benefit to surfaces contacted therewith. Such pro-perfumes are also especially easy to suspend in liquid detergent products.

### CROSS REFERENCE TO RELATED APPLICATIONS

This application claims priority under 35 U.S.C. §119(e) to U.S. Provisional Application Serial No. 60/290,280, filed May 11, 2001 (Attorney Docket No. CM2565FP).

### TECHNICAL FIELD

The present invention relates to pro-perfume compositions, in particular for use in cleaning or fabric treatment products. Such pro-perfume compositions impart sustained release of a multi-odor perfume profile, i.e., a freshness benefit, on surfaces like fabrics, in particular dry fabrics, which have been treated with such products.

### BACKGROUND OF THE INVENTION

Perfumed products are well-known in the art. However, consumer acceptance of such perfumed products like laundry and cleaning products is determined not only by the performance achieved with these products but also by the aesthetics associated therewith. The perfume components are therefore an important aspect of the successful formulation of such commercial products.

It is also desired by consumers for treated surfaces like fabrics to maintain the pleasing fragrance over time. Indeed, perfume additives make such compositions more aesthetically pleasing to the consumer, and in some cases the perfume imparts a pleasant fragrance to surfaces, like fabrics, treated therewith. However, the amount of perfume carried-over from an aqueous laundry or cleaning bath onto fabrics is often marginal and does not last long on the surface. Furthermore, fragrance materials are often very costly and their inefficient use in laundry and cleaning compositions and ineffective delivery to surfaces like fabrics results in a very high cost to both consumers and laundry and cleaning manufacturers. Industry, therefore, continues to seek with urgency for more efficient and effective fragrance delivery in laundry and cleaning products, especially for improvement in the provision of long-lasting fragrance to the surfaces like fabrics.

Recently, a new class of materials, namely the amine reaction product of a compound containing a primary and/or secondary amine functional group and an active ketone or aldehyde containing component, have found increasing use in the domestic treatment of fabrics in order to provide long lasting perfume release on the laundered fabric. Disclosure of such compounds can be found, for example, in PCT Patent Application Nos. WO 00/02991; WO 00/02981; WO 00/02987 and WO 00/02982.

However, notwithstanding the advances in the art, there is still a need for identifying pro-perfumes which deliver the benefit of a multi-odor profile to substrates and surfaces treated therewith. There is also still a need to provide pro-perfumes which can be stably incorporated into a wide variety of cleaning and fabric treatment products, especially those in the form of liquid detergent compositions.

Accordingly, it is an object of the present invention to provide pro-perfume compositions which can impart a multi-odor benefit profile to surfaces such as fabrics which have been contacted with such pro-perfumes It is a further object of the present invention to provide pro-perfume compositions which can be effectively suspended and stably incorporated within liquid detergent compositions.

### SUMMARY OF THE INVENTION

The present invention provides pro-perfume compositions which can be incorporated into cleaning products, e.g., detergent compositions, or fabric treatment products. Such pro-perfume compositions comprise the reaction product of a primary and/or secondary amine compound with a combination of a perfume ketone and a relatively “bulky” perfume aldehyde.

The amine compound is one which has an Odor Intensity Index of less than that of a 1% solution of methylanthranilate in dipropylene glycol. The perfume aldehyde is one which has relatively high boiling point and relatively high molecular weight. The ketone/aldehyde combination contains perfume ketone and perfume aldehyde in a weight ratio of from about 95:5 to 25:75.

The cleaning and fabric treatment products containing such pro-perfume compositions impart a sustained, multi-odor perfume or freshness benefit to the surfaces treated with such products. Such products in liquid form are also especially stable and easy to prepare.

### DETAILED DESCRIPTION OF THE INVENTION

The essential components of the pro-perfume reaction product compositions herein are the primary and/or secondary amine compound and the combination of perfume ketones and aldehydes which reacts with the amine compound. Each of these components as well as composition preparation and cleaning or fabric treatment products containing such pro-perfume compositions, are described in detail as follows:

Amine Compound

The amine compound used to form the pro-perfume compostions herein is a primary and/or secondary amine. The term “primary and/or secondary amine”, means for purposes of this invention a component which carries at least one primary and/or secondary amine and/or amide function. Of course, one amine compound may carry both primary and secondary amine moieties thereby enabling a reaction with several aldehydes and/or ketones.

The primary amine and/or secondary amine compounds used in this invention are generally ones characterized by having an Odor Intensity Index of less than that of a 1% solution of methylanthranilate in dipropylene glycol.

Odor Intensity Index Method

Odor Intensity Index is a value determined by expert graders who evaluate test chemicals for odor when such the pure chemicals are diluted at 1% in dipropylene glycol (DPG), odor-free solvent used in perfumery. This concentration percentage is representative of typical usage levels. Smelling strips, or so called “blotters”, were are dipped in test solutions and presented to the expert panellist for evaluation. Expert panellists are assessors trained for at least six months in odor grading and whose gradings are checked for accuracy and reproducibility versus a reference on an on-going basis. For each amine compound, a panellist is presented two blotters: one reference (Me Anthranilate, unknown from the panellist) and the test sample. The panellist is asked to rank both smelling strips on the 0-5 odor intensity scale, 0 being no odor detected, 5 being very strong odor present.

Results:

The following represents Odor Intensity Index of some amine compounds suitable for use in the present invention and according to the above procedure. In each case, numbers are arithmetic averages among 5 expert panellists and the results are statistically significantly different at 95% confidence level:

| | |
|---|---|
| Methylanthranilate 1% (reference) | 3.4 |
| Ethyl-4-aminobenzoate (EAB) 1% | 0.9 |
| 1,4-bis-(3-aminopropyl)-piperazine (BNPP) 1% | 1.0 |

A wide variety of primary and/or seconday amine compounds which have the requisite Odor Intensity Index characteristics can be used to prepare the pro-perfume compositions of this invention. A general structure for a primary amine compound useful in this invention is as follows:

B—(NH2)ₙ;

wherein B is a carrier material, and n is an index of value of at least 1. Compounds containing a secondary amine group have a structure similar to the above excepted that the compound comprises one or more —NH— groups instead of —NH2. Further, the compound structure may also have one or more of both —NH2 and —NH— groups. Typically the amine compounds of this general type are relatively viscous materials as are the pro-perfume reaction products made therefrom.

Suitable B carriers include both inorganic and organic carrier moieties. By “inorganic carrier”, it is meant a carrier which is comprised of non- or substantially non-carbon based backbones.

Preferred primary and/or secondary amines, utilizing inorganic carriers, are those selected from mono or polymers or organic-organosilicon copolymers of amino derivatised organo silane, siloxane, silazane, alumane, aluminum siloxane, or aluminum silicate compounds. Typical examples of such carriers are: organosiloxanes with at least one primary amine moiety like the diaminoalkylsiloxane [H2NCH2(CH3)2Si]O, or the organoaminosilane (C6H5) 3SiNH2 described in: Chemistry and Technology of Silicone, W. Noll, Academic Press Inc. 1998, London, pp 209, 106).

Preferred primary and/or secondary amines, utilizing organic carriers, are those selected from aminoaryl derivatives, polyamines, amino acids and derivatives thereof, substituted amines and amides, glucamines, dendrimers, polyvinylamines and derivatives thereof, and/or copolymer thereof, alkylene polyamine, polyaminoacid and copolymer thereof, cross-linked polyaminoacids, amino substituted polyvinylalcohol, polyoxyethylene bis amine or bis aminoalkyl, aminoalkyl piperazine and derivatives thereof, bis (amino alkyl) alkyl diamine linear or branched, and mixtures thereof.

Preferred aminoaryl derivatives are the amino-benzene derivatives including the alkyl esters of 4-amino benzoate compounds, and more preferably selected from ethyl-4-amino benzoate, phenylethyl-4-aminobenzoate, phenyl-4-aminobenzoate, 4-amino-N′-(3-aminopropyl)-benzamide, and mixtures thereof.

Polyamines suitable for use in the present invention are polyethyleneimines polymers, poly[oxy(methyl-1,2-ethanediyl)], α-(2-aminomethylethyl)-ω-(2-aminomethyl-ethoxy)- (=C.A.S No. 9046-10-0); poly[oxy(methyl-1,2-ethanediyl)], α-hydro-)-ω-(2-aminomethylethoxy)-, ether with 2-ethyl-2-(hydroxymethyl)-1,3-propanediol (=C.A.S. No. 39423-51-3); commercially available under the tradename Jeffamines T-403, D-230, D-400, D-2000; 2,2′,2″-triaminotriethylamine; 2,2′-diamino-diethylamine; 3,3′-diamino-dipropylamine, 1,3 bis aminoethyl-cyclohexane commercially available from Mitsubishi and the C12 Sternamines commercially available from Clariant like the C12 Sternamin(propylenamine)ₙ with n=¾, and mixtures thereof. Preferred polyamines are polyethyleneimines commercially available under the tradename Lupasol like Lupasol FG (MW 800), G20wfv (MW 1300), PR8515 (MW 2000), WF (MW 25000), FC (MW 800), G20 (MW 1300), G35 (MW 1200), G100 (MW 2000), HF (MW 25000), P (MW 750000), PS (MW 750000), SK (MW 2000000), SNA (MW 1000000). Of these, the most preferred include Lupasol HF or WF (MW 25000), P (MW 750000), PS (MW 750000), SK (MW 2000000), 620wfv (MW 1300) and PR 1815 (MW 2000).

Preferred amino acids for use herein are selected from tyrosine, tryptophane, lysine, glutamic acid, glutamine, aspartic acid, arginine, asparagine, phenylalanine, proline, glycine, serine, histidine, threonine, methionine, and mixture thereof, most preferably selected from tyrosine, tryptophane, and mixture thereof. Preferred amino acid derivatives are selected from tyrosine ethylate, glycine methylate, tryptophane ethylate, and mixture thereof.

Preferred substituted amines and amides for use herein are selected from nipecotamide, N-coco-1,3-propenediamine; N-oleyl-1,3-propenediamine; N-(tallow alkyl)-1,3-propenediamine; 1,4-diamino cyclohexane; 1,2-diamino-cyclohexane; 1,12-diaminododecane, and mixtures thereof.

Other primary amine compounds suitable for use herein are the glucamines, preferably selected from 2,3,4,5,6-pentamethoxy-glucamine; 6-acetylglucamine, glucamine, and mixture thereof.

Also preferred compounds are the polyethylenimine and/or polypropylenimine dendrimers and the commercially available Starburst® polyamidoamines (PAMAM) dendrimers, generation G0-G10 from Dendritech and the dendrimers Astromols®, generation 1-5 from DSM being DiAminoButane PolyAmine DAB (PA)x dendrimers with x=2ⁿ×4 and n being generally comprised between 0 and 4.

Polyamino acid is one suitable and preferred class of amino-functional polymer. Polyaminoacids are compounds which are made up of amino acids or chemically modified amino acids. They can contain alanine, serine, aspartic acid, arginine, valine, threonine, glutamic acid, leucine, cysteine, histidine lysine, isoleucine, tyrosine, asparagine, methionine, proline, tryptophan, phenylalanine, glutamine, glycine or mixtures thereof. In chemically modified amino acids, the amine or acidic function of the amino acid has reacted with a chemical reagent. This is often done to protect these chemical amine and acid functions of the amino acid in a subsequent reaction or to give special properties to the amino acids, like improved solubility. Examples of such chemical modifications are benzyloxycarbonyl, aminobutyric acid, butyl ester, pyroglutamic acid. More examples of common modifications of amino acids and small amino acid fragments can be found in the Bachem, 1996, Peptides and Biochemicals Catalog.

Preferred polyamino acids are polylysines, polyarginine, polyglutamine, polyasparagine, polyhistidine, polytryptophane or mixtures thereof. Most preferred are polylysines or polyamino acids where more than 50% of the amino acids are lysine, since the primary amine function in the side chain of the lysine is the most reactive amine of all amino acids.

The preferred polyamino acid has a molecular weight of 500 to 10,000,000, more preferably between 2,000 and 25,000.

The polyamino acid can be cross linked. The cross linking can be obtained for example by condensation of the amine group in the side chain of the amino acid like lysine with the carboxyl function on the amino acid or with protein cross linkers like PEG derivatives. The cross linked polyamino acids still need to have free primary and/or secondary amino groups left for reaction with the active ingredient.

The preferred cross linked polyamino acid has a molecular weight of 20,000 to 10,000,000, more preferably between 200,000 and 2,000,000.

The polyamino acid or the amino acid can be co-polymerized with other reagents like for instance with acids, amides, acyl chlorides. More specifically with aminocaproic acid, adipic acid, ethylhexanoic acid, caprolactam or mixture thereof. The molar ratio used in these copolymers ranges from 1:1 (reagent/amino acid (lysine)) to 1:20, more preferably from 1:1 to 1:10.

The polyamino acid like polylysine can also be partially ethoxylated.

Examples and supply of polyaminoacids containing lysine, arginine, glutamine, asparagine are given in the Bachem 1996, Peptides and Biochemicals catalog.

The polyaminoacid can be obtained before reaction with the active ingredient, under a salt form. For example polylysine can be supplied as polylysine hydrobromide. Polylysine hydrobromide is commercially available from Sigma, Applichem, Bachem and Fluka.

Examples of suitable amino functional polymers containing at least one primary and/or secondary amine group for the purpose of the present invention are:

Polyvinylamine with a MW of about 300-2.10E6;

Polyvinylamine alkoxylated with a MW of about 600, 1200 or 3000 and an ethoxylation degree of 0.5;

Polyvinylamine vinylalcohol—molar ratio 2:1, polyvinylaminevinylformamide—molar ratio 1:2 and polyvinylamine vinylformamide—molar ratio 2:1;

Triethylenetetramine, diethylenetriamine, tetraethylenepentamine;

Bis-aminopropylpiperazine;

Polyamino acid (L-lysine/lauric acid in a molar ratio of 10/1), Polyamino acid (L-lysine/aminocaproic acid/adipic acid in a molar ratio of 5/5/1), ), Polyamino acid (L-lysine/aminocaproic acid/ethylhexanoic acid in a molar ratio of 5/3/1) Polyamino acid (polylysine-cocaprolactam); Polylysine; Polylysine hydrobromide; cross-linked polylysine,

amino substituted polyvinylalcohol with a MW ranging from 400-300,000;

polyoxyethylene bis [amine] available from e.g. Sigma;

polyoxyethylene bis [6-aminohexyl] available from e.g. Sigma;

N,N′-bis-(3-aminopropyl)-1,3-propanediamine linear or branched (TPTA); and

1,4-bis-(3-aminopropyl) piperazine (BNPP).

The more preferred compounds are selected from ethyl-4-amino benzoate, polyethyleneimine polymers commercially available under the tradename Lupasol like Lupasol HF, P, PS, SK, SNA, WF, G20wfv and PR8515; the diaminobutane dendrimers Astramol®, polylysine, cross-linked polylysine, N,N′-bis-(3-aminopropyl)-1,3-propanediamine linear or branched; 1,4-bis-(3-aminopropyl) piperazine, and mixtures thereof. Even most preferred compounds are those selected from ethyl-4-amino benzoate, polyethyleneimine polymers having a molecular weight grater than 200 daltons including those commercially available under the tradename Lupasol like Lupasol HF, P, PS, SK, SNA, WF, G20wfv and PR8515; polylysine, cross-linked polylysine, N,N′-bis-(3-aminopropyl)-1,3-propanediamine linear or branched, 1,4-bis-(3-aminopropyl) piperazine, and mixtures thereof.

Advantageously, such most preferred primary and/or secondary amine compounds also provide fabric appearance benefit, in particular color appearance benefit, thus providing a resulting amine reaction product with the properties of fabric appearance benefit, deposition onto the surface to be treated, and delayed release of the active as well as release of the perfume composition. Further, when the primary and/or secondary amine compound has more than one free primary and/or secondary amine group, several different active ingredients (aldehyde and/or ketone) can be linked to the amine compound.

Of course, an excess of the primary and/or secondary amine compound may also be used in the pro-perfume compositions herein as is, i.e. without having been reacted with an aldehyde and/or ketone perfume ingredient, but with a benefit agent, as described hereinafter, like a perfume composition which is entrapped or embedded within the primary and/or secondary amine compound. Moreover, the primary and/or secondary amine compound may also be reacted with compounds other than the perfume ketone or aldehyde, like acyl halides, like acetylchloride, palmytoyl chloride or myristoyl chloride, acid anhydrides like acetic anhydride, alkylhalides or arylhalides to do alkylation or arylation, aldehydes or ketones not used as perfume ingredients like formaldehyde, glutaraldehyde, unsaturated ketones, aldehydes or carboxylic acids like 2-decylpropenoic acid, propenal, propenone to form reaction products with the desired physical properties.

Perfume Ketone/Perfume Aldehyde Combination

To form the pro-perfume compositions herein, the primary and/or secondary amine compund as described hereinbefore is reacted with a combination of perfume compounds which essentially comprises a perfume ketone component and a perfume aldehyde component. In such a combination, the weight ratio of ketone to aldehyde will generally range form about 95:1 to 25:75, more preferably from about 95:1 to 60:40. Stated another way the ketone/aldehyde combination will generally comprise from about 25% to 95% by weight of the perfume ketone component, more preferably from about 40% to 95% of the perfume ketone component. The perfume ketone and perfume aldehyde components of this combination are described in greater detail as follows:

A) Perfume Ketone Component

The perfume ketones utilized in the pro-perfume compositions herein can comprise any material which is chemically a ketone, which can react with the amine moiety of the amine compound and which can impart a desirable odor or freshness benefit to surfaces which have been treated with the pro-perfume composition. The perfume ketone component can, of course, comprise more than one ketone, i.e., mixtures of ketones. Preferably, the perfume ketone is selected from buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-Ionone, Beta-Ionone, Gamma-Methyl so-called Ionone, Fleuramone, Dihydrojasmone, Cis-Jasmone, Iso-E-Super, Methyl-Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane, 6,7-Dihydro-1,1,2,3,3-Pentamethyl-4(5H)-Indanone, 4-Damascol, Dulcinyl or Cassione, Gelsone, Hexalon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, Tetrameran, hedione, and mixtures thereof.

More preferably, from the above-mentioned compounds, the preferred perfume ketones are selected from Alpha Damascone, Delta Damascone, Iso Damascone, Carvone, Gamma-Methyl-Ionone, Iso-E-Super, 2,4,4,7-Tetramethyl-oct-6-en-3-one, Benzyl Acetone, Beta Damascone, Damascenone, methyl dihydrojasmonate, methyl cedrylone, hedione, and mixtures thereof.

B) Perfume Aldehyde Component

The perfume aldehyde component of the pro-perfume compositions herein can comprise any perfume material which is chemically an aldehyde, which can, like the perfume ketone component, react with the amino moiety of the amine compound and which can also impart a desirable odor or freshness benefit to surfaces which have been treated with the pro-perfume composition. As with the perfume ketone component, the perfume aldehyde component can comprise a single individual aldehyde or mixtures of two or more perfume aldehydes. In addition, the perfume aldehyde component must comprise aldehydes which are relatively “bulky.” By bulky, it is meant that the perfume aldehyde will have relatively high molecular weight and have a relatively high boiling point. For purposes of this invention, high molecular weight perfume aldehydes are those having a boiling point greater than about 225° C. Further, for purposes of this invention, high molecular weight perfume aldehydes are those with a molecular weight greater than about 150.

More preferably the perfume aldehydes used herein will comprise materials which have a boiling point above 250° C. and a Clog P greater than 3. Clog P is a commonly known calculated measure as defined in the following references “Calculating log P_{oct} from Structures”; Albert Leo (Medicinal Chemistry Project, Pomona College, Claremont Calif. USA. Chemical Reviews, Vol. 93, number 4, June 1993; as well as from Comprehensive Medicinal Chemistry, Albert Leo, C. Hansch, Ed. Pergamon Press: Oxford, 1990, Vol. 4, p.315; and Calculation Procedures for molecular lipophilicity: a comparative Study, Quant. Struct. Act. Realt. 15, 403-409 (1996), Raymund Mannhold and Karl Dross.

Suitable perfume aldehyde materials for use in the pro-perfumes herein, whether by themselves or as part of a perfume aldehyde mixture, include adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxy0hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, 1-p-menthene-q-carboxaldehyde, citral, lilial and mixtures thereof.

More preferred perfume aldehydes are selected from citral, 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-l-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, trans-2-nonenal, lilial, trans-2-nonenal, and mixture thereof.

C) Optional Ingredients/Benefit Agents

In addition to the essential combination of perfume ketones and perfume aldehydes which is reacted with the amine compund to form the pro-perfumes herein, these pro-perfume compositions can contain a wide variety of optional ingredients. Such optional ingredients can either be reacted with the amine compound as are the essential perfume ingredients or they can be simply physically admixed with and entrapped in the essential pro-perfume components. These optional ingredients are referred to herein as benefit agents since they can provide a beneficial effect on a treated surface, like fabric, upon subsequent contact of the treated surface with water or humidity. Hence, the benefit agent may be selected from a flavor ingredient, a pharmaceutical ingredient, a biocontrol ingredient, an additional perfume composition which may or may not include perfumes which are aldehydes or ketones, a refreshing cooling ingredient and mixtures thereof.

Typically, the benefit agent can comprise from 10 to 90%, preferably from 30 to 85%, more preferably from 45 to 80% by weight of the pro-perfume component.

Flavor ingredients include spices, flavor enhancers that contribute to the overall flavor perception.

Pharmaceutical ingredients include drugs.

Biocontrol ingredients include biocides, antimicrobials, bactericides, fungicides, algaecides, mildewcides, disinfectants, antiseptics, insecticides, vermicides, and plant growth hormones.

Typical antimicrobials which can be carried by the pro-perfume compositions include amine oxide surfactants, photo-activated bleaches, chlorhexidine diacetate, glutaraldehyde, cinnamon oil and cinnamaldehyde, citric acid, decanoic acid, lactic acid, maleic acid, nonanoic acid, polybiguanide, propylene glycol, cumene sulfonate, eugenol, thymol, benzalkonium chloride, geraniol, and mixtures thereof. Preferred are compounds which can react with the amine compund or the carrier material thereof.

Typical insect and/or moth repellants are perfume ingredients, such as citronellal, citral, N,N diethyl meta toluamide, Rotundial, 8-acetoxycarvotanacetone, and mixtures thereof. Other examples of insect and/or moth repellant for use herein are disclosed in U.S. Pat. Nos. 4,449,987, 4,693,890, 4,696,676, 4,933,371, 5,030,660, 5,196,200, and “Semio Activity of Flavor and Fragrance molecules on various Insect Species”, B. D. Mookherjee et al., published in Bioactive Volatile Compounds from Plants, ASC Symposium Series 525, R. Teranishi, R. G. Buttery, and H. Sugisawa, 1993, pp. 35-48.

As indicated, the benefit agent may also comprise a perfume composition made of mixture of perfume ingredients including or not the above mentioned aldehyde or ketone perfumes. This composition is then entrapped within the pro-perfume component by mixing. By such means, a more fully complete perfume formulation can then be deposited onto the contacted surface.

Typical of these ingredients include fragrant substance or mixture of substances including natural (i.e., obtained by extraction of flowers, herbs, leaves, roots, barks, wood, blossoms or plants), artificial (i.e., a mixture of different nature oils or oil constituents) and synthetic (i.e., synthetically produced) odoriferous substances. Such materials are often accompanied by auxiliary materials, such as fixatives, extenders, stabilizers and solvents. These auxiliaries are also included within the meaning of “perfume”, as used herein. Typically, perfumes are complex mixtures of a plurality of organic compounds.

Suitable perfumes are disclosed in U.S. Pat. No. 5,500,138, said patent being incorporated herein by reference.

Examples of perfume ingredients useful in the perfume compositions include, but are not limited to, amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma.

Additional examples of fragrance materials include, but are not limited to, orange oil; lemon oil; grapefruit oil; bergamot oil; clove oil; dodecalactone gamma; methyl-2-(2-pentyl-3-oxo-cyclopentyl) acetate; beta-naphthol methylether; methyl-beta-naphthylketone; coumarin; 4-tert-butylcyclohexyl acetate; alpha,alpha-dimethylphenethyl acetate; methylphenylcarbinyl acetate; cyclic ethyleneglycol diester of tridecandioic acid; 3,7-dimethyl-2,6-octadiene-1-nitrile; ionone gamma methyl; ionone alpha; ionone beta; petitgrain; methyl cedrylone; 7-acetyl-1,2,3,4,5,6,7,8-octahydro-1,1,6,7-tetramethyl-naphthalene; ionone methyl; methyl-1,6,10-trimethyl-2,5,9-cyclododecatrien-1-yl ketone; 7-acetyl-1, 1,3,4,4,6-hexamethyl tetralin; 4-acetyl-6-tert-butyl-1,1-dimethyl indane; benzophenone; 6-acetyl-1,1,2,3,3,5-hexamethyl indane; 5-acetyl-3-isopropyl-1,1,2,6-tetramethyl indane; 1-dodecanal; 7-hydroxy-3,7-dimethyl octanal; 10-undecen-1-al; iso-hexenyl cyclohexyl carboxaldehyde; formyl tricyclodecan; cyclopentadecanolide; 16-hydroxy-9-hexadecenoic acid lactone; 1,3,4,6,7,8-hexahydro-4,6,6,7,8,8-hexamethylcyclopenta-gamma-2-benzopyrane; ambroxane; dodecahydro-3a,6,6,9a-tetramethylnaphtho-[2,1b]furan; cedrol; 5-(2,2,3-trimethylcyclopent-3-enyl)-3-methylpentan-2-ol; 2-ethyl-4-(2,2,3-trimethyl-3-cyclopenten-1-yl)-2-buten-1-ol; caryophyllene alcohol; cedryl acetate; para-tert-butylcyclohexyl acetate; patchouli; olibanum resinoid; labdanum; vetivert; copaiba balsam; fir balsam-hydroxycitronellal and indol; phenyl acetaldehyde and indol.

More examples of perfume components are geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methylether; isolongifolanone; aubepine nitrile; aubepine; heliotropine; eugenol; vanillin; diphenyl oxide; hydroxycitronellal ionones; methyl ionones; isomethyl ionomes; irones; cis-3-hexenol and esters thereof; indane musk fragrances; tetralin musk fragrances; isochroman musk fragrances; macrocyclic ketones; macrolactone musk fragrances; ethylene brassylate. Also suitable herein as perfume ingredients of the pro-perfume compositions are the so-called Schiff base. Schiff-Bases are the condensation of an aldehyde perfume ingredient with an anthranilate. A typical description can be found in U.S. Pat. No. 4,853,369. Typical of Schiff bases are selected from Schiffs base of 4-(4-hydroxy-4-methylpentyl)-3-cyclohexene-1-carboxaldehyde and methyl anthranilate; condensation products of: hydroxycitronellal and methyl anthranilate; 4-(4-hydroxy-4-methyl pentyl)-3-cyclohexene-1-carboxaldehyde and methyl anthranilate; Methyl Anthranilate and HydroxyCitronellal commercially available under the tradename Aurantiol; Methyl Anthranilate and Methyl Nonyl Acetaldehyde commercially available under the tradename Agrumea; Methyl Anthranilate and PT Bucinal commercially available under the tradename Verdantiol; Methyl anthranilate and Lyral commercially available under the tradename Lyrame; Methyl Anthranilate and Ligustral commercially available under the tradename Ligantral; and mixtures thereof.

Preferably, the perfume ingredients and/or compositions useful in the present invention compositions are substantially free of halogenated materials and nitromusks.

More preferably, the perfume compounds are characterised by having a low Odor Detection Threshold. Such Odor Detection Threshold (ODT) should be lower than 1 ppm, preferably lower than 10 ppb—measured at controlled Gas Chromatography (GC) conditions such as described here below. This parameter refers to the value commonly used in the perfumery arts and which is the lowest concentration at which significant detection takes place that some odorous material is present. Please refer for example in “Compilation of Odor and Taste Threshold Value Data (ASTM DS 48 A)”, edited by F. A. Fazzalari, International Business Machines, Hopwell Junction, N.Y. and in Calkin et al., Perfumery, Practice and Principles, John Willey & Sons, Inc., page 243 et seq (1994). For the purpose of the present invention, the Odor Detection Threshold is measured according to the following method:

The gas chromatograph is characterized to determine the exact volume of material injected by the syringe, the precise split ratio, and the hydrocarbon response using a hydrocarbon standard of known concentration and chain-length distribution. The air flow rate is accurately measured and, assuming the duration of a human inhalation to last 0.02 minutes, the sampled volume is calculated. Since the precise concentration at the detector at any point in time is known, the mass per volume inhaled is known and hence the concentration of material. To determine the ODT of a perfume material, solutions are delivered to the sniff port at the back-calculated concentration. A panelist sniffs the GC effluent and identifies the retention time when odor is noticed. The average over all panelists determines the threshold of noticeability. The necessary amount of analyte is injected onto the column to achieve a certain concentration, such as 10 pph, at the detector. Typical gas chromatograph parameters for determining odor detection thresholds are listed below.

GC: 5890 Series II with FID detector

7673 Autosampler

Column: J&W Scientific DB-1

Length 30 meters ID 0.25 mm film thickness 1 micron

Method:

Split Injection: 17/1 split ratio

Autosampler: 1.13 microliters per injection

Column Flow: 1.10 mL/minute

Air Flow: 345 mL/minute

Inlet Temp. 245° C.

Detector Temp. 285° C.

Temperature Information

Initial Temperature: 50° C.

Rate: 5C/minute

Final Temperature: 280° C.

Final Time: 6 minutes

Leading assumptions: 0.02 minutes per sniff

GC air adds to sample dilution

Examples of such preferred perfume components are those selected from: 2-methyl-2-(para-iso-propylphenyl)-propionaldehyde, 1-(2,6,6-trimethyl-2-cyclohexan-1-yl)-2-buten-1-one and/or para-methoxy-acetophenone. Even more preferred are the following compounds having an ODT <10 ppb measured with the method described above: undecylenic aldehyde, undecalactone gamma, heliotropin, dodecalactone gamma, p-anisic aldehyde, para hydroxy-phenyl-butanone, cymal, benzyl acetone, ionone alpha, p.t.bucinal, damascenone, ionone beta, methyl-nonyl ketone, methyl heptine carbonate, linalool, indol, cis-3-hexenyl salicylate, vanillin, methyl isobutenyl tetrahydropyran, ethylvanillin, coumarin, ethyl methyl phenyl glycidate, eugenol, methylanthranilate, iso eugenol, beta naphtol methyl ester, herbavert, lyral, allyl amyl glycolate, dihydro iso jasmonate, ethyl-2-methylbutyrate, nerol, and phenylacetaldehyde. Most preferably the optional perfume ingredients comprise at least 5%, more preferably at least 10% of the compositions herein.

Most preferably, the perfume ingredients are those as described in WO 96/12785 on page 12-14. Even most preferred are those perfume compositions comprising at least 10%, preferably 25%, by weight of perfume ingredient with an ClogP of at least 2.0, preferably of at least 3.0 and boiling point of at least 250° C. Still another preferred perfume composition is a composition comprising at least 20%, preferably 35%, by weight of perfume ingredient with an ClogP at least 2.0, more preferably of at least 3.0 and boiling point of less than or equal to 250° C.

Pro-Perfume Composition Preparation

The pro-perfume compositions herein can be prepared by simply admixing the amine compound and the perfume ketone/perfume aldehyde combination under conditions which are sufficient to bring about reaction of these components. Frequently this admixing is carried out using high shear agitation. Temperatures of from about 40° C. to 65° C. may be utilized. Additional benefit agents may also be added to the reaction mixture. The reaction mechanism involving the reaction of the amine compound with the ketone/aldehyde perfume combination is described in greater detail in PCT Application No. WO 00/02982, which publication is incorporated herein by reference.

Typically equimolar amount of the reactants can be employed. On a weight basis, reactant amounts can vary widely, ranging from 5:1 to 1:5 for the two essential components. (amine compund and ketone/aldehyde combination). These two components do not have to be added to the reaction mixture simultaneously. The ketone and aldehyde components may instead by added sequentially. To form a suitable reaction medium, the reactants may also be admixed with one or more components of the cleaning or fabric treatment products into which the pro-perfume compositions herein will eventually be formulated.

As indicated, the resulting pro-perfume reaction products are relatively viscous materials. Frequently the viscosity of the amine compound reaction products will be greater than about 1000 cPs, more preferably greater than about 500,000 cPs, and even more preferably greater than about 1,000,000 cPs.

Cleaning and Fabric Treatment Products

The pro-perfume compositions of the present invention can be incorporated into a wide variety of cleaning products and fabric treatment products. Such products include both laundry and cleaning compositions which are typically used for laundering fabrics and cleaning hard surfaces such as dishware, floors, bathrooms, toilet, kitchen and other surfaces in need of a delayed release of perfume ketone and aldehyde. Accordingly, by laundry and cleaning compositions, these are to be understood to include not only detergent compositions which provide fabric cleaning benefits, but also compositions such as hard surface cleaning which provide hard surface cleaning benefit.

Products in which the pro-perfumes herein can be incorporated also include fabric treatment products such as fabric softeners or conditioners. Such products do not necessarily impart a cleaning benefit to fabrics treated therewith.

Preferred as products in which the pro-perfumes herein can be incorporated are are those laundry and fabric treatment, e.g., softener, compositions which result in contacting of the pro-perfume with fabric.

The effectiveness of the delivery to treated surfaces of the pro-perfumes herein can be quantified by means of a parameter called the Dry Surface Odor Index. Such a parameter is fully described in PCT Application No. WO 00/02982, which publication is incorporated by reference herein. Preferably, the pro-perfume compositions herein which are incorporated into cleaning and fabric treatment products will provide a Dry Surface Odor Index of more than about 5 and preferably at least about 10.

In general, the pro-perfume compositions herein can be incorporated into cleaning or fabric treatment products herein at levels which range from about 0.005% to 5% by weight, more preferably from about 0.02% to 0.5% by weight. For cleaning products, the pro-perfume will generally be incorporated at concentrations of from about 0.005% to 2% by weight, along with from about 1% to 50% by weight of a detersive surfactant. For fabric treatment products, the pro-perfume will generally be incorporated at I concentrations of from about 0.005% to 5% by weight, along with from about 1% to 50% by weight of a fabric softening or treating agent. The cleaning and fabric treatment products containing the pro-perfumes herein can comprise a wide variety of additional adjuvants which are conventional for use in products of these types. Extensive disclosure of such conventional adjuvants can be found in PCT Patent Application Nos. WO 00/02982 and WO 00/02987, which publications are incorporated herein by reference.

The cleaning and treatment products which contain the pro-perfumes herein may take a variety of physical forms including liquids, gels or foams in aqueous or nonaquous form, granular form or tablet form. An especially preferred form for products of this type is a liquid detergent composition, e.g., a heavy duty liquid (HDL) detergent for fabric laundering. Pro-perfumes comprising the reaction product of higher molecular weght, higher viscosity amines and an aldehyde or ketone have typically been incorporated into liquid detergent compositions using a suspending agent, such as silicone emulsifiers, to preserve both the chemical and physical stability of the pro perfume in such liquid products. It has been discovered, however, that when the pro-perfume comprises the reaction product of an amine with a combination of both a perfume ketone and a “bulky” perfume aldhyde of the type herein utilized, the suspending agent is not needed. Thus the pro-perfumes herein can be incorporated into liquid detergent products which are substantially free of silicone-based suspending agents.

Preparation of the pro-perfumes herein and their incorporation into certain types of cleaning products can be illustrated by the following examples:

### EXAMPLE I

A pro-perfume composition is prepared which comprises the reaction product of the polyethyleneimine Lupasol WF (MW=25,000) with a combination of delta-damascone and alpha-n-hexyl cinnamic aldehyde. To prepare such a pro-perfume, 1368 grams of delta-damascone and 72 grams of the cinnamic aldehyde are weighed into a vessel and mixed. Separately a mixture of the Luposol (960 grams) and the ethoxylated nonionic surfactant Neodol 23-5 (3600 grams) is prepared. This is done by heating the Neodol to 50° C. and by then adding the Lupasol to the hot Neodol. The Lupasol/Neodol mixture is then added to the damascone/cinnamic aldehyde mixture which is then stirred for about one hour. The resulting reaction product can be added as a pro-perfume composition to a wide variety of cleaning and fabric treament product types.

A variety of detergent compositions are prepared having ethe compositions shown in the following Examples II through VI. In these examples the abbreviated component identifications have the following meanings:

| | |
|---|---|
| LAS: | Sodium linear C₁₂ alkyl benzene sulphonate |
| CFAA: | C₁₂-C₁₄ alkyl N-methyl glucamide |
| HEDP: | Hydroxyethane dimethylene phosphonic acid |
| DETPMP: | Diethylene triamine penta (methylene phosphonic |
| | acid), marketed by Monsanto under the Tradename |
| | Dequest 2060 |
| TEPAE | Tetreaethylenepentaamine ethoxylate |
| PVP | Polyvinylpyrrolidone polymer |
| PVNO | Polyvinylpyridine-N-Oxide, with an average molecular |
| | weight of 50,000. |
| Brightener | Disodium 4,4′-bis(2-sulphostyryl)biphenyl and/or |
| | Disodium 4,4′-bis(4-anilino-6-morpholino-1.3.5- |
| | triazin-2-yl) stilbene- 2:2′-disulfonate. |
| Suds Suppressor- | 25% paraffin wax Mpt 50° C., 17% hydrophobic |
| | silica, 58% paraffin oil Granular suds suppressors 12% |
| | Silicone/silica, 18% stearyl alcohol, 70% starch in |
| | granular form |
| Enzymes: | Protease, amylase, cellulase and/or lipase |
| SRP | Anionically end capped poly esters. |
| MEA | Monoethanolamine |
| SCS | Sodium Cumene Sulfonate |

### EXAMPLE II

Liquid Detergent Composition

A heavy duty liquid (HDL) detergent composition is prepared containing the pro-perfume composition of Example I. Such a liquid detergent composition has the following formula:

| Component | Wt. % |
|---|---|
| C₁₂₋₁₅ alkyl ether (2.5) sulfate | 19.0 |
| C₁₂₋₁₃ alkyl ethoxylate (9.0) | 2.00 |
| C₁₂₋₁₄ glucose amide | 3.50 |
| Citric Acid | 3.00 |
| C₁₂₋₁₄ Fatty Acid | 2.00 |
| MEA | to pH 8 |
| Ethanol | 3.41 |
| Propanediol | 6.51 |
| Borax | 2.5 |
| Dispersant | 1.18 |
| Na Toluene Sulfonate | 2.50 |
| Pro-Perfume Composition* of Example I | 0.3% |
| Dye, Brighteners, Enzymes, Preservatives, Suds | Balance |
| Suppressor, Other Minors, Water | |
| | 100% |

*Including the Neodol

### EXAMPLE III

The following liquid detergent formulations are prepared according to the present invention:

| | A | B | C | D | E |
|---|---|---|---|---|---|
| LAS | 11.5 | 9.0 | — | 4.0 | — |
| C25E2.5S | — | 3.0 | 18.0 | — | 16.0 |
| C45E2.25S | 11.5 | 3.0 | — | 16.0 | — |
| C23E9 | — | 3.0 | 2.0 | 2.0 | 1.0 |
| C23E7 | 3.2 | — | — | — | — |
| CFAA | — | — | 5.0 | — | 3.0 |
| TopPalmKernel Fatty Acid | 2.0 | — | 2.0 | 0.5 | 2.0 |
| Citric (50%) | 6.5 | 1.0 | 2.5 | 4.0 | 2.5 |
| Ca and/or Ca formate | 0.6 | 0.7 | 0.2 | 0.05 | 0.05 |
| SCS | 4.0 | 1.0 | 3.0 | 1.2 | — |
| Borate | 0.6 | — | 3.0 | 2.0 | 3.0 |
| Na hydroxide | 6.0 | 2.0 | 3.5 | 4.0 | 3.0 |
| Ethanol | 2.0 | 1.0 | 4.0 | 4.0 | 3.0 |
| 1,2 Propanediol | 3.0 | 2.0 | 8.0 | 8.0 | 5.0 |
| Monoethanolamine | 3.0 | 1.5 | 1.0 | 2.5 | 1.0 |
| TEPAE | 2.0 | — | 1.0 | 1.0 | 1.0 |
| Pro-Perfume of Ex. I | 0.1 | 0.02 | 0.1 | 0.01 | 0.05 |
| Enzymes | 0.03 | 0.01 | 0.03 | 0.02 | 0.02 |
| SRP | 0.2 | — | 0.1 | — | — |
| DTPA | — | — | 0.3 | — | — |
| PVNO | — | — | 0.3 | — | 0.2 |
| Brightener | 0.2 | 0.07 | 0.1 | — | — |
| Suds suppressor | 0.04 | 0.02 | 0.1 | 0.1 | 0.1 |

| | |
|---|---|
| Miscellaneous and water | Balance to 100% |

### EXAMPLE IX

Heavy duty liquid fabric cleaning compositions in accordance with the invention are prepared as follows:

| | A | B |
|---|---|---|
| LAS acid form | — | 25.0 |
| Citric acid | 5.0 | 2.0 |
| 25AS acid form | 8.0 | — |
| 25AE2S acid form | 3.0 | — |
| 25AE7 | 8.0 | — |
| CFAA | 5 | — |
| DETPMP | 1.0 | 1.0 |
| Fatty acid | 8 | — |
| Oleic acid | — | 1.0 |
| Ethanol | 4.0 | 6.0 |
| Propanediol | 2.0 | 6.0 |
| Pro-Perfume (Ex. I) | 0.10 | 0.05 |
| Coco-alkyl dimethyl | — | 3.0 |
| hydroxy ethyl ammonium | | |
| chloride | | |
| Smectite clay | — | 5.0 |
| PVP | 2.0 | — |

| | |
|---|---|
| Water/Minors | Up to 100% |

### EXAMPLE V

Heavy-duty liquid fabric cleaning compositions in accordance with the invention are prepared as follows:

| | A | B | C |
|---|---|---|---|
| C25AES | 18.0 | 15.0 | 14.0 |
| LAS | 5.8 | 5.0 | 4.0 |
| C₈₋₁₀ Amine | 1.4 | 2.0 | — |
| Nonionic 24-7 | 2.8 | 2.0 | 3.0 |
| Citric acid | 2.5 | 3.0 | 3.0 |
| Fatty acid | 8.5 | 3.0 | 3.0 |
| Enzymes | 0.02 | 0.02 | 0.006 |
| Boric acid | 2.0 | 2.0 | 2.0 |
| Ethoxylate tetraethylene pentaimine | 0.9 | 1.0 | 1.0 |
| Polyethylene imine ethoxylated | 0.7 | — | 1.0 |
| DETPMP | 0.3 | — | — |
| HEDP | 0.35 | — | — |
| Ethanol | 1.0 | 3.0 | 3.0 |
| 1,2,propanediol | 8.0 | 4.0 | 5.0 |
| MEA | 9.8 | 2.0 | 2.0 |
| Na Cumene Sulfonate | 2.0 | — | — |
| Suds suppressors | 0.25 | 0.01 | 0.01 |
| Pro-Perfume of Example I | 0.07 | 0.03 | 0.01 |

| | |
|---|---|
| Minors (perfumes, brighteners, . . .) and | Up to 100% |
| water | |

### EXAMPLE VI

Granular Detergent Composition

A heavy duty granular detergent (HDG) composition is prepared containing the pro-perfume composition of Example I. Such a granular detergent composition has the following formula:

| Component | Wt. % |
|---|---|
| C₁₂ Linear alkyl benzene sulfonate | 9.31 |
| C₁₄₋₁₅ alkyl sulfonate | 12.74 |
| Zeolite Builder | 27.79 |
| Sodium Carbonate | 27.31 |
| PEG 4000 | 1.60 |
| Dispersant | 2.26 |
| C₁₂₋₁₃ alkyl ethoxylate (E9) | 1.5 |
| Sodium Perborate | 1.03 |
| Soil Release Polymer | 0.41 |
| Enzymes | 0.59 |
| Pro-Perfume of Example I | 0.1 |
| Brightener, Suds Suppressor, Other Minors, Moisture, Sulfate | Balance |
| | 100% |

What is claimed is:

1. A pro-perfume composition, comprising the reaction product of a primary and/or secondary amine compound with a combination of a perfume ketone component and a high boiling point, high molecular weight perfume aldehyde component; said combination having a weight ratio of ketone to aldehyde of from about 95:5 to about 25:75; and said amine compound having an Odor Intensity Index of less than that of a 1% solution of methylanthranilate in dipropylene glycol.

2. A composition according to claim 1 wherein the amine compound is selected from ethyl-4-amino benzoate, polyethyleneimine polymers; diaminobutane dendrimers Astramol®, polylysine, cross-linked polylysine, N,N′-bis-(3-aminopropyl)-1,3-propanediamine linear or branched; 1,4-bis-(3-aminopropyl) piperazine, and mixtures thereof.

3. A composition according to claim 1 wherein the perfume ketone component is selected from Alpha Damascone, Delta Damascone, Iso Damascone, Carvone, Gamma-Methyl-Ionone, Iso-E-Super, 2,4,4,7-Tetramethyl-oct-6-en-3-one, Benzyl Acetone, Beta Damascone, Damascenone, methyl dihydrojasmonate, methyl cedrylone, hedione, and mixtures thereof.

4. A composition according to claim 1 wherein the perfume aldehyde component is selected from citral, 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, trans-2-nonenal, lilial, trans-2-nonenal, and mixtures thereof.

5. A composition according to claim 1 wherein the pro-perfume composition further comprises an additional perfume composition.

6. A pro-perfume composition, comprising the reaction product of an amine compound with a combination of a perfume ketone component and a high molecular weight perfume aldehyde component, wherein
A) said amine compound is selected from polyethyleneimines having a molecular weight greater than 200 daltons;
B) said combination comprises from about 25% to about 95% by weight of the combination of the perfume ketone component and from about 5% to about 75% by weight of the combination of the perfume aldehyde component; and
C) said aldehyde component has a boiling point above 250° C. and a C Log P of greater than 3.

7. A pro-perfume composition according to claim 6 wherein:
A) said amine compound is selected from LUPASOL FG, LUPASOL WF, LUPASOL P, LUPASOL HF, LUPASOL G20WFV AND LUPASOL PR8515:
B) said perfume ketone component is selected from Damascone, alpha-Damascone, beta-Damascone, delta-Damascone, iso-Damascone and mixtures thereof; and
C) said perfume aldehyde component is selected from lilial, alpha-n-hexylcinnamic aldehyde, alpha-n-amylcinnamic aldehyde, cymal, lyral butylcinnamic aldehyde and mixtures thereof.

8. A cleaning or fabric treatment product comprising from about 0.005% to about 5% by weight of a pro-perfume composition according to claim 1.

9. A cleaning composition comprising from about 1% to about 50% by weight of a detersive surfactant and from about 0.005% to about 2% by weight of a pro-perfume composition according to claim 6.

10. A cleaning composition according to claim 9 which is in the form of a liquid detergent composition.

11. A fabric treatment composition comprising from about 1% to about 50% by weight of a fabric softening or treatment agent and from about 0.005% to about 5% of a pro-perfume composition according to claim 6.

12. A pro-perfume composition according to claim 1 wherein the composition is suitable for incorporation into cleaning or fabric treatment products.

13. A pro-perfume composition according to claim 6 wherein the composition is suitable for incorporation into cleaning or fabric treatment products.

14. A cleaning composition comprising from about 1% to about 50% of a detersive surfactant and from about 0.005% to about 2% by weight of a pro-perfume composition comprising the reaction product of:
i) a primary and/or secondary amine compound having an Odor Intensity Index of less than that of a 1% solution of methylanthranilate in dipropylene glycol; with
ii) a combination of a perfume ketone component and a high boiling point, high molecular weight perfume aldehyde component, said combination having a weight ratio of ketone to aldehyde of from about 95:5 to about 25:75.

15. A cleaning composition according to claim 14 wherein;
a) said amine compound is selected from polyethyleneimines having a molecular weight greater than 200 daltons;
b) said aldehyde component has a boiling point above 250° C. and a C Log P of greater than 3.

16. A cleaning composition according to claim 2 wherein the cleaning composition comprises an additional perfume composition.

17. A cleaning composition according to claim 6 wherein the cleaning composition comprises an additional perfume composition.

18. A cleaning composition according to claim 14 wherein the cleaning composition comprises an additional perfume composition.

19. A cleaning composition according to claim 14 which is in the form of a liquid detergent composition.

20. A fabric treatment composition comprising from about 1% to about 50% by weight of a fabric softening or treatment agent and from about 0.005% to about 5% of a pro-perfume composition according to claim 1.
